(19) United States
(12) Patent Application Publication
(10) Pub. No.: US 2024/0345054 A1
(43) Pub. Date: Oct. 17, 2024

(54) Method of Screening Antioxidants

(71) Applicant: Johnson & Johnson Consumer Inc., Skillman, NJ (US)

(72) Inventors: Nikoleta Batchvarova, Skillman, NJ (US); Allison Rush, Spring House, PA (US)

(21) Appl. No.: 18/579,625

(22) PCT Filed: Jul. 14, 2022
(86) PCT No.: PCT/IB2022/056512

§ 371 Date: Jan. 16, 2024

Related U.S. Application Data
(60) Provisional application No. 63/222,039, filed on Jul. 15, 2021.

(51) Int. Cl. G01N 33/15; A61K 31/665; A61P 39/06

(52) U.S. Cl. CPC G01N 33/15; A61K 31/665; A61P 39/06

(57) ABSTRACT

Methods of screening types and amounts of antioxidants, determining effectiveness of the antioxidants, preparing compositions including effective amounts of the antioxidants (and uses thereof), and reducing the formation of clogged pores and acne are provided. Methods include the formation of test samples in a solvent system (optionally with one or more bacterial metabolites) including one or more antioxidants in amounts to be evaluated for effectiveness against control samples without antioxidants. The samples are exposed to an oxidation-inducing element (such as UVA rays) and the amount of oxidation byproduct is evaluated. Based on the effectiveness of the antioxidant(s) in the amount(s) tested, formulations can be prepared to include the one or more antioxidants in the amount(s) effective to reduce oxidation of the oxidizable component, preventing and reducing the formation of the oxidation byproduct. The formulations can be used, for example, to reduce the formation of clogged pores and acne.

### CROSS-REFERENCE TO RELATED APPLICATIONS

This application claims the benefit of priority to U.S. Provisional Patent Appln. Ser. No. 63/222,039, filed on Jul. 15, 2021, the contents of which are incorporated herein by reference in their entirety.

### FIELD

The present disclosure relates to methods of screening components for efficacy in mitigating oxidation, and includes methods of preparing compositions and compositions prepared that are effective in mitigating oxidation.

### BACKGROUND

Oxidation of components is a natural phenomenon that occurs due to exposure of a number of elements, including, for example, the presence of ultraviolet radiation. Certain biological components can be susceptible to oxidation, and sometimes such oxidation can have potential negative implications. Oxidation of ingredients in compositions can also occur, which can damage the composition and/or reduce its effectiveness.

One result of oxidation of a component in biological fluid is acne. Acne is a complex multifactorial skin disease that affects the quality of life and self-confidence of consumers. Clear skin remains an unmet need for many individuals, in part because some of the pathophysiological factors associated with acne development that negatively affect the health of their pores are not sufficiently treated with skincare products that target the symptoms of acne, i.e., with keratolytic and antimicrobial ingredients. While treatment of acne is important and provides benefits to the user, it would be helpful to provide compositions to be applied to the face or body that include elements that mitigate or reduce the likelihood of acne generation/recurrence.

Oxidation of squalene, and particularly UV-induced squalene oxidation, is a factor associated with early inflammatory events in human skin, where the inflammatory events can lead to development of an acne lesion and/or other detrimental effects on the skin such as clogged pores, inflammatory disease, hyperpigmentation, aging, and cutaneous malignancy. Other sources of reactive oxygen species resulting in oxidation include, for example, bacteria, chemicals, pollution or toxins, ionizing or nonionizing radiation, inflammation, mitochondrial metabolism, and enzymes. Mitigation of the oxidation can be useful in reducing the likelihood of acne formation, exacerbation, and length of disease as well as the development of clogged pores in an acne-free oily skin. Some useful components that mitigate the oxidation include antioxidants, however, not all antioxidants work the same, and some work better than others as it relates to effects on oily and/or acne skin. It would therefore be useful to be able to identify effective and useful antioxidants to be used in compositions, and prepare compositions including these antioxidants. In addition, it would be useful to be able to identify the most efficient amount of antioxidant to include in such compositions, to avoid either including too little or too much of a certain component or to provide formulations with extended-release and long-acting anti-oxidant benefits.

The present disclosure provides methods of determining efficacy of antioxidants in reducing or mitigating UV-induced oxidation of a component, and includes identification of an efficient amount of such antioxidant in reducing oxidation of that component. The present disclosure further includes methods of forming compositions including effective antioxidants in efficient amounts as well as compositions including effective ingredients in efficient amounts.

### SUMMARY

Methods of determining effectiveness of antioxidants, preparation of compositions including effective amounts of the antioxidants (and uses thereof), and methods of reducing the formation of acne are provided. Formulations include one or more antioxidants in an amount effective to reduce oxidation of an oxidizable component, preventing and reducing the formation of an oxidation byproduct. The method includes formation of test samples in a solvent system, optionally with one or more bacterial metabolites, with test samples including one or more antioxidants in amounts to be evaluated for effectiveness against control samples lacking antioxidants. Exposure to an oxidation-inducing element, such as UVA rays, is performed on each sample, and the amount of oxidation byproduct is evaluated. Depending upon the effectiveness of the antioxidant(s) in the amount(s) tested, a formulation including the antioxidant or antioxidants can be prepared, desirably in the amount demonstrated to be effective. Methods of preparing compositions to reduce the formation of clogged pores and acne are provided, using the method of determining effectiveness of an antioxidant or antioxidants in reducing the formation of malondialdehyde (MDA) by squalene exposed to UVA radiation. Methods of reducing acne by using compositions prepared by the aforementioned method are provided.

Methods of screening one or more antioxidants in the reduction of oxidation of an oxidizable component are provided. The method includes preparing one or more test samples including the oxidizable component, the one or more antioxidants, a solvent, and optionally one or more bacterial metabolites: preparing one or more control samples without the one or more antioxidants and comprising the oxidizable component, the solvent, and optionally the one or more bacterial metabolites: performing an oxidation test on each of the samples: measuring an amount of an oxidation byproduct of each of the samples; and determining a level of oxidation mitigation by comparing the measured amount of the oxidation byproduct in the one or more test samples to the measured amount of the oxidation byproduct in the one or more control samples.

In certain embodiments, the oxidizable component can include squalene.

In certain embodiments, the samples can include the oxidizable component in an amount of from about 1% (v/v) to about 30% (v/v). In certain embodiments, the samples can include the oxidizable component in an amount of about 30% (v/v) or less.

In certain embodiments, the solvent can include dimethyl sulfoxide, propylene glycol, 1-butanol, or combinations thereof. In certain embodiments can include dimethyl sulfoxide in an amount of from about 5% (v/v) to 15% (v/v), propylene glycol in an amount of from about 5% (v/v) to about 15% (v/v), and 1-butanol in an amount of from about 70% (v/v) to about 90% (v/v). In certain embodiments, the solvent can be a blend of dimethyl sulfoxide, propylene glycol, and 1-butanol in a volumetric ratio of about 1:1:10.

In certain embodiments, the oxidation byproduct can include malondialdehyde (MDA).

In certain embodiments, the measured amount of the oxidized byproduct in the one or more test samples can be at least about 10% less than the measured amount of the oxidized byproduct in the one or more control samples. In certain embodiments, the measured amount of the oxidized byproduct in the one or more test samples can be at least about 25% less than the measured amount of the oxidized byproduct in the one or more control samples.

In certain embodiments, the oxidation test can include exposing the samples to an oxidation-inducing element. In certain embodiments, the oxidation-inducing element can include UV radiation.

In certain embodiments, the samples can include the one or more bacterial metabolites in an amount of from about 0.01 nM to about 2.0 nM. In certain embodiments, the one or more bacterial metabolites can include protoporphyrin IX, coproporphyrin I, coproporphyrin III, or combinations thereof.

In certain embodiments, the one or more antioxidants can include vitamins, natural extracts containing phenolic antioxidant compounds, synthetic antioxidants, or combinations thereof.

In certain embodiments, the amount of the oxidation byproduct can be measured by fluorometric analysis.

Methods of determining efficacy of one or more antioxidants in reducing or mitigating oxidation of an oxidizable component are provided. The method includes selecting a type and an amount of the oxidizable component as a target: selecting one or more antioxidants for testing against the target: selecting a solvent: optionally selecting one or more bacterial metabolites: combining the oxidizable component, the one or more antioxidants, the solvent, and optionally the one or more bacterial metabolites, to form a test sample: performing an oxidation test on the test sample: measuring an amount of an oxidation byproduct of the test sample, and determining a level of oxidation mitigation by comparing the measured amount of the oxidation byproduct of the test sample to a control sample without the one or more antioxidants.

In certain embodiments, the oxidizable component can include squalene.

In certain embodiments, the samples can include the oxidizable component in an amount of from about 1% (v/v) to about 30% (v/v).

In certain embodiments, the solvent can include dimethyl sulfoxide, propylene glycol, 1-butanol, or combinations thereof. In certain embodiments, the solvent can include dimethyl sulfoxide in an amount of from about 5% (v/v) to 15% (v/v), propylene glycol in an amount of from about 5% (v/v) to about 15% (v/v), and 1-butanol in an amount of from about 70% (v/v) to about 90% (v/v).

In certain embodiments, the oxidation byproduct can include malondialdehyde (MDA).

In certain embodiments, the oxidation test can include exposing the samples to a predetermined amount and a predetermined time of UV radiation. In certain embodiments, the predetermined time of UV radiation exposure can be from about 10 J/cm2 to about 30 J/cm2.

In certain embodiments, the measured amount of the oxidization byproduct in the test sample can be less than the measured amount of the oxidization byproduct in the control sample by at least 5%.

In certain embodiments, the method can further include preparing a composition including the one or more antioxidants determined to be effective in reducing or mitigating oxidation of the oxidizable component.

Methods of using a composition including one or more antioxidants are provided. The method includes applying the composition to a surface including an oxidizable component. The composition is effective at reducing oxidation of the oxidizable component.

In certain embodiments, the composition can be a topical composition. In particular embodiments, the composition can be a moisturizer or a sunscreen.

In certain embodiments, the composition can reduce the formation of clogged pores or acne.

### DETAILED DESCRIPTION

Oxidation can happen to many components as a result of natural phenomena. One cause of oxidation is exposure to ultraviolet radiation, such as UV rays from the sun. Exposure of certain levels of UV radiation for a certain length of time can result in oxidation of a component. There are components that occur within the body that are susceptible to oxidation, and the oxidation of such components can have negative implications. Further, oxidizable components can be useful as ingredients in formulations, such as skin care compositions or other useful compositions, but oxidation of the ingredient can have negative implications, such as reducing the efficacy of the formulation.

The present disclosure relates to methods of reducing oxidation of components, and in particular, reducing oxidation of components through the addition of an effective type of, and efficient level of, antioxidant(s). Antioxidants are known to reduce oxidation of components, however, the type and amount of antioxidant required to reduce oxidation can vary. The amount and type can vary depending upon the type of oxidizable component, or the amount of oxidizable component, or the reactive oxygen species responsible for oxidation, or the level of activity that causes oxidation (such as, for example, UV radiation or certain bacteria) that the oxidizable component is exposed to. Through the present disclosure, a user can understand the most effective and efficient antioxidant to use for a particular oxidizable component, and can generate compositions to reduce oxidation, and can use compositions in a way that reduces or mitigates oxidation of the component.

As used herein, the “Oxidizable Component” refers to an ingredient or material that is susceptible to oxidation, and in particular, susceptible to oxidation due to the presence of ultraviolet radiation. As used herein, an “antioxidant” refers to an ingredient or blend of ingredients that has an effect of inhibiting and reducing the level of or altogether eliminating oxidation of the Oxidizable Component. Through the present disclosure, a composition can be prepared that includes an antioxidant that is the proper type of antioxidant in the proper level of antioxidant in a way that provides efficient and effective reduction in oxidation of the Oxidizable Component. As used herein, “efficient” reduction in oxidation does not require complete elimination of oxidation, but rather reduces the amount of oxidation in a statistically significant manner when compared to the level of oxidation that would occur in the absence of that antioxidant. The level of oxidation can be determined by measuring the resulting peroxides that are generated by oxidation of the Oxidizable Component.

The present disclosure is directed to effectively reducing the level of oxidation in one or more Oxidizable Components. Oxidizable Components can include, for example, biological components, such as squalene (and other lipids), protein, and DNA. Oxidizable Components can include ingredients in compositions, such as vitamins, fragrances, natural extracts, and any other labile ingredients intended to deliver benefits to the user and/or provide stability, aesthetics, and/or performance to the formulation.

In one aspect of the present disclosure, the present disclosure is directed to reduction in the oxidation of a biological component, such as squalene, and can be directed to reduction in the generation/recurrence of acne and/or clogged pores in a user. Squalene is an abundant sebaceous lipid in human skin and is present in amounts of about 12-20% (w/w) of the sebum composition. Squalene is an endogenous sebaceous lipid in the skin that is most susceptible to oxidation. It is now understood that inflammation plays an early role in the development and/or recurrence of acne, and oxidation of squalene is now considered to be a probable cause of inflammation that can result in the development/recurrence of inflammatory and non-inflammatory acne lesions. In fact, squalene peroxides (which are developed through oxidation), malondialdehyde (MDA, which is a lipid/squalene peroxidation byproduct), and other biomarkers associated with oxidative stress (i.e., reduced glutathione, adenosine deaminase, superoxide dismutase activity, and catalase activity) have been observed at significantly higher levels in the sebum or skin scrapings of acne-suffering subjects compared to subjects that do not suffer from acne. It has been seen that the levels of MDA and adenosine deaminase in such skin scrapings are associated with acne severity, where the higher level of MDA and adenosine deaminase is correlated with higher likelihood of acne or higher severity of acne.

Furthermore, topical application of squalene peroxides has been shown to induce acne lesions (i.e., “comedones”) in a rabbit ear model. While non-oxidized squalene is a clear, non-viscous liquid that can be easily excreted from the pores, oxidized squalene forms a semisolid yellow wax which can contribute to the clogging of pores (i.e., pilosebaceous units), resulting in inflammation and, in some instances, acne breakouts. MDA can also contribute to pore clogging by cross-linking to proteins and altering protein rigidity in the pilosebaceous unit. It is therefore desired to reduce the formation of MDA and/or adenosine deaminase in and on the surface of skin of an individual.

While the example set forth above and described herein refers to squalene as the Oxidizable Component, it is understood that the present disclosure can and does relate to reduction in oxidation of any other Oxidizable Component, including those identified above. Reduction in oxidation of Oxidizable Components has a number of benefits, including biological benefits as well as the benefit of maintaining efficacy and stability of products and formulations. By way of example, it is contemplated that if an Oxidizable Component is used in a topical formulation, it can be susceptible to the UV rays after applied to the skin, and use of one or more proper antioxidants can be useful in protecting such oxidation.

With regard to reduction in squalene oxidation, the present disclosure can include methods of improving the skin of a user, and in particular, mitigating the risk of acne and/or clogged pore generation and/or recurrence in the skin of the user. As noted above, squalene is found in skin sebum, and therefore, application of a skin topical formulation that includes a proper antioxidant in a proper amount can help reduce UV-induced squalene oxidation. Prevention of such oxidation, or reduction in the level of such oxidation, can help prevent early inflammation in the pores of the skin that leads to hyperkeratinization in the pore and acne development. This method relates to methods of determining efficacious antioxidants, as well as the use levels of antioxidants, where the antioxidants can be incorporated into a topical formulation to be applied by the user.

Therefore, protection of the squalene in the skin's sebum, especially in oily skin and reduction in oxidation of the squalene can provide a significant pore health benefit, including reducing the likelihood of acne formation. Antioxidants can help by reducing the oxidation of squalene, however, not every antioxidant is useful as a skin-applied ingredient. Further, some antioxidants can provide benefits at lower levels than other antioxidants, so the amount of antioxidant required for effective reduction in oxidation can vary widely. Reliable and consistent evaluation of the antioxidant or antioxidants to be used in a topical formulation is therefore important to providing a product that gives skin health benefit to the user. While in vivo methods of testing antioxidants are helpful, they are costly and not convenient for screening multiple ingredients at the same time, and they require willing subjects for such testing.

In vitro methods of evaluating antioxidants have been used in the past, however, previous models and characterizations of antioxidants have failed in certain areas. In particular, some do not employ a consistent solvent system to solubilize the Oxidizable Component and also test various antioxidants. In particular, some previous methods do not use the same solvent for different components, and therefore can run the risk of convoluting results since some solvents can have intrinsic antioxidant effects. Further, other previous methods do not incorporate hydrophilic and lipophilic antioxidants to enable relative comparisons of antioxidants. Still other previous models do not employ clinically relevant dose conditions, such as those that would take into account the native squalene levels for oily or dry skin in relation to the product levels applied to skin and levels of porphyrins, bacterial metabolites present in skin which act as photocatalysts and therefore contribute to squalene oxidation. It would be useful to perform an in vitro test that takes into account biologically relevant levels of squalene, in addition to bacterial metabolites present on skin, and UV-dose levels that replicate sun exposure levels.

The present disclosure solves these and other problems associated with current testing of antioxidant efficacy and efficiency, specifically with regard to oxidation of the particular Oxidizable Component. Further, the methods described herein can result in preparation of formulations that include a desired type and amount of antioxidant to reduce oxidation of an Oxidizable Component found within that formulation. Further, the methods described herein can result in preparation of compositions that include a desired type and amount of antioxidant to reduce or mitigate oxidation of a biological Oxidizable Component, including skin, where the compositions can be applied to the skin of a user. Compositions such as moisturizers, lotions, sunscreens, and other leave-on compositions are contemplated herein, as are rinse-off compositions such as cleansers, where the rinse-off composition can leave a residual amount of product, including antioxidant, on the skin after rinsing.

Compositions made according to the present disclosure that are intended to achieve a certain effect can include certain benefit agents associated with that effect. For example, a sunscreen can include one or more sun filter agents, while a moisturizing agent can include one or more ingredients effective to provide moisturization to the skin. Compositions can include one or more antioxidants in the amount determined by the present disclosure, in addition to the benefit agent(s) associated with the intended benefit of the composition.

The present disclosure relates to and includes a method of screening types and amounts of antioxidants to mitigate UV-induced oxidation of an Oxidizable Component, and further allows for preparation of a composition including the type and amount of antioxidant screened for. The method includes and comprises the use of the Oxidizable Component, in addition to one or more solvents, at least one bacterial metabolite, such as protoporphyrin IX, and at least one target antioxidant.

The Oxidizable Component can be any component that is susceptible to oxidation and which is desired to have a reduction of oxidation. As noted above, any Oxidizable Component can be used, and in some embodiments, the Oxidizable Component is a biological material, and can include squalene. As such, an amount of Oxidizable Component is useful in the test methods, and the Oxidizable Component can be obtained from any source. If squalene is the Oxidizable Component target, the squalene can be mammalian in nature, such as from human or animal samples. Alternatively, the squalene can be synthetically produced. In addition, the squalene can be extracted from sebum, in the case of natural squalene, or it can be left within sebum, such that the entire composition of the extracted sebum is tested.

The method includes one or more solvents, and can incorporate the same solvent for each target antioxidant or Oxidizable Component to be tested. Any desired solvent can be used as desired. It can be desired that the solvent includes at least one of dimethyl sulfoxide, propylene glycol, and 1-butanol, and can include all three in a blended solvent mixture. In some aspects, the blend includes a higher level of 1-butanol than the dimethyl sulfoxide and propylene glycol, respectively, and can include, for example dimethyl sulfoxide (in an amount of from about 5% to about 15% v/v), propylene glycol (in an amount of from about 5% to about 15% v/v), and 1-butanol (in an amount of from about 70% to about 90% v/v). In one exemplary embodiment, the solvent is a blend of dimethyl sulfoxide, propylene glycol, and 1-butanol in a volumetric ratio of about 1:1:10.

The method further includes one or more endogenous bacterial metabolites present in skin/comedones. The benefit of using (a) bacterial metabolite(s) is that they act as photocatalysts and contribute to the magnitude of squalene oxidation as well as represent real life conditions present in the pores and on the skin. Oily skin and acne skin in particular triggers the proliferation of certain bacteria and induces the production of such metabolites. Suitable bacterial metabolites include, for example, porphyrins, such as protoporphyrin IX, coproporphyrin I, and coproporphyrin III, and combinations thereof.

Any desired antioxidant can be tested through this method, including, for example, synthetic antioxidants and antioxidants extracted from natural entities (i.e., plants, vegetables, fruits). It is noted, however, that certain antioxidants have known properties that can be affected by the stability in the formulation. For example, certain antioxidants, such as Vitamin C and Vitamin E, are labile and therefore their effectiveness as an antioxidant can be potentiated when used in combination with other antioxidants. The method can test one single antioxidant or can test multiple antioxidants in combination with each other.

The method of the present disclosure is beneficial in that it allows the user to employ clinically relevant dose conditions (e.g., the ratio of squalene levels in different types of skin as compared to typical product coverage on skin), as well as exposure conditions (i.e., such as the amount and length of UV radiation) to determine efficacious antioxidants and use levels to incorporate into a formulation. The method allows for manipulation of the dose conditions to provide efficacy to different skin of different types, ages, gender, hormonal state, etc. It also allows for adjustment to different exposure conditions to provide for efficacy under various real-life situations.

UV radiation exposure conditions can be changed depending upon desired consumer use, as well as to account for regions having different environmental conditions. For example, it can be desired to test for efficacy of an antioxidant for a certain Oxidizable Component under conditions where a user would be exposed to the sun's UV rays for a prolonged period of time. This can be desired if the product is to be applied to this skin of the user and the user would plan to be exposed to the sun's rays for an extended period of time. A slow-release of antioxidants might be most applicable to such situation. In addition, different geographic regions are susceptible to different levels of UV radiation, and this method can account for the differences in UV exposure depending upon geographic location. Pollution components, such as ozone, particulate matter and smoke, can also play a role in the oxidation, and therefore one or more of these additional components can be incorporated into the test to give additional real-world examples.

One exemplary benefit to the present innovative process of determining efficacy and efficiency of an antioxidant on a particular Oxidizable Component is that it allows for one solvent to achieve solubilization of the Oxidizable Component tested, as well as both hydrophilic and lipophilic antioxidants, and also provides for simple assay methods to perform the analysis of byproducts formed by oxidation. In particular, the method described herein can be useful in identifying one or more byproducts of oxidation, for example, as noted above, one byproduct of squalene oxidation which is malondialdehyde (MDA). The presence and amount of MDA that is formed after a certain amount and length of UV radiation exposure can help determine the efficacy of the type and amount of antioxidant tested under those conditions. The result of this process identifies useful and effective antioxidants under targeted conditions, and also helps determine the amount of such antioxidant required to achieve an efficient amount of oxidation mitigation of the Oxidizable Component under those conditions.

The method incorporates an assay where at least one target antioxidant is tested for efficacy of an Oxidizable Component, as compared to a control sample of the Oxidizable Component that has vehicle with no added antioxidant. The target sample and the control sample are exposed to the same conditions, which can be modified as needed to give desired results. As noted above, certain conditions such as the amount of Oxidizable Component, and/or the length of time of exposure to UV-radiation, and/or the level of UV-radiation can be modified as desired. The level of mitigation of UV-induced oxidation of the Oxidizable Component for a tested antioxidant can be determined by comparing (1) the amount of oxidation byproduct (e.g., MDA) formed after exposure to UV radiation in the absence of the target antioxidant to (2) the amount of oxidation byproduct (e.g., MDA) formed after exposure to UV radiation including the target antioxidant in the tested amount. It can be desired to include two groups falling under category (1), where one is UV-stimulated to show the magnitude of oxidation induced in the absence of an antioxidant, and another is not UV-stimulated, which can be used as a baseline. This allows the user to quickly and easily determine the efficacy and efficiency of the target antioxidant under the desired conditions. Further details of the test method are described below.

As a result of the present innovation, compositions including a desired amount and desired type of antioxidant can be prepared, where the desired amount of antioxidant is effective in mitigation of UV-induced oxidation of the Oxidizable Component. In some aspects, compositions can be prepared that include an efficient and effective amount of an antioxidant to reduce or eliminate oxidation of an Oxidizable Component ingredient found in that composition, such as when the Oxidizable Component is squalene and the oxidized result is MDA. In other aspects, compositions can be prepared that include an efficient and effective amount of an antioxidant to reduce or eliminate oxidation of an Oxidizable Component that is found on the skin of a user (such as squalene). Further, methods of using compositions including the desired amount of antioxidant can be achieved, providing benefits as described above to the user.

While full elimination of the Oxidizable Component's oxidized byproduct (e.g., MDA formed by squalene oxidation) would represent complete prevention of oxidation, the present method and composition does not require full elimination of the oxidized byproduct to be considered effective. It is desired, however, that the amount of the oxidized byproduct generated in a sample demonstrate a statistically significant reduction compared to a sample without antioxidant. In some aspects, the reduction in oxidation can be at least 25% less than the amount of the oxidized byproduct generated in a sample without antioxidant present, or at least 15% less than the amount of the oxidized byproduct generated in a sample without antioxidant present, or at least 10% less than the amount of the oxidized byproduct generated in a sample without antioxidant present. It is noted that the effectiveness of an antioxidant can also depend upon the amount of antioxidant tested, and therefore the most efficient amount of antioxidant to achieve such effectiveness can also be determined. For example, one antioxidant present at different levels in a composition can provide a different amount of oxidation reduction depending upon the amount of the antioxidant, and therefore “efficiency” of the antioxidant can be dependent upon the amount of antioxidant present. The level of oxidation mitigation is determined by comparing the oxidized byproduct generation level in the absence of the antioxidant compared to the oxidized byproduct generation level with the antioxidant present. When testing a plurality of antioxidants, particularly against a control sample which has no antioxidant, it can be desired to compare the level of oxidized byproduct to determine those which show a statistically significant reduction compared to the level of oxidized byproduct in the absence of an antioxidant. The percentage reduction can be at least 25%, 15% or 10% less than the control sample, and depending upon the desired outcome, any or all reduction can be deemed sufficient. Based on the proposed model the comparative strength of an antioxidant versus others might be determined based on comparison of the achieved level of reduction of oxidation and the respective amount of antioxidant used to do that.

A method of determining effectiveness and efficiency of an antioxidant in reducing oxidation of a target Oxidizable Component is described herein. The target Oxidizable Component is determined, and the amount of Oxidizable Component to be tested is determined. The amount of the Oxidizable Component should remain consistent through each sample tested. For example, when testing squalene, the amount of the squalene tested can be about 1% to about 30% (v/v), or about 5% to about 15% (v/v), or about 9.7% to about 10% (v/v) in each sample. This amount can be increased or reduced depending upon the real-world amount that is experienced. For oily skin, for example, the amount of squalene can be as high as about 30.0% (v/v) (i.e., about 30.0% (v/v) or less) in each sample, while for dry skin, the amount of squalene can be as low as 1.5% (v/v) (i.e., about 1.5% (v/v) or less) in each sample. This modification allows for proper evaluation of the antioxidant effect that would be found in an actual user. If other Oxidizable Components are used, the amount tested can reflect the amount found in nature, or the amount to be used in a formulation or composition.

One or more target antioxidants are selected for testing, where the amount of antioxidant can be modified for each sample tested. Any number of antioxidants can be tested in any desired amount, but when determining and making relative comparisons of the effectiveness and efficiency of the antioxidants, the amount of the Oxidizable Component should remain consistent for each sample tested, regardless of the type of target antioxidant or the amount of target antioxidant tested.

A method of determining the effective and efficient level of antioxidant for the target Oxidizable Component is described herein. The method includes one or more of the steps of choosing an Oxidizable Component to serve as the target, choosing one or more antioxidants to test against the target, choosing a solvent, optionally choosing a bacterial metabolite, combining the materials for testing, choosing a level and length of time for exposure to UV rays, and subjecting samples to the UV rays, as will be described below.

The Oxidizable Component is selected to serve as the target. The Oxidizable Component can be any material that is susceptible to oxidation, and in some case, the Oxidizable Component can be a biological component, such as squalene. The amount of Oxidizable Component is also determined, which should reflect the actual amount of Oxidizable Component that would be used or exists in nature. For example, if 0.1 grams/L of an Oxidizable Component would be used in a formulation, then it can be desired to test 0.1 g/L of that Oxidizable Component. Further, if it is known that a certain amount of biological component exists in nature, then that amount can be used.

For example, in the case of squalene in human sebum, it can be desired to use an amount that would be representative of the “Casual Sebum Level”. The Casual Sebum Level is the amount of sebum typically present on the skin surface, and the proportion of squalene in sebum (i.e., about 12-20% w/w). Casual Sebum Levels can vary from individual to individual, and can differ based upon factors such as gender, ethnicity, age, anatomical location, stress, diet, and skin condition(s), with the typical range being about 35-320 μg/cm² for dry to oily facial skin. The average casual sebum level is about 70 to about 150 μg/cm² for the “T-zone” area (i.e., forehead, nose, and chin) of adults with normal facial skin.

Squalene is typically about 10-15% (w/w) of the sebaceous lipid composition, whereas up to about 20% has been observed in acne skin. In this regard, the representative amount of squalene used in the present innovative method can range from about 4.2 μg/cm² (representative of dry/non-acne skin, 12% squalene, Casual Sebum Level=35 μg/cm²) to about 63.2 μg/cm² (representative of highly oily, acne skin, 20% squalene, Casual Sebum Level=320 μg/cm²). Given these levels, a representative amount of squalene for acne skin that is oily but not excessively oily can be from about 20 to about 40 μg/cm², or about 25-30 μg/cm². A squalene level of 25 μg/cm² represents a level that is 20% squalene in sebum, with a Casual Sebum Level of 125 μg/cm². This level is halfway between the normal skin level of 100 μg/cm² and 150 μg/cm², the lower range for hyperseborrhea, and is close to average levels measured in the T-zone of acne subjects (i.e., 132 μg/cm²). Any levels of squalene desired can be used, depending upon the correlation to the skin type being evaluated.

Clinically relevant dose conditions can be employed in this model. The amount of antioxidant added to each sample can be representative of the intended use level in the formulation, assuming a 1 mg/cm² product application to skin, which is a typical amount of product that consumers apply to skin. Of course, any level of product application can be considered for the method, such as 2 mg/cm². For the tests conducted, a 30 μL sample volume was chosen for squalene. The density of squalene was 0.74 g/mL, equating to 22.3 mg of squalene per sample. Considering the representative amount of squalene that is used (25 μg/cm²), the representative weight of the product is 892.8 mg per sample (1 mg/cm² product on skin is 40 times higher than the amount of squalene). This product amount was not incorporated into the sample, instead it was used to calculate the amount of antioxidant included in the sample, based on the intended use level (i.e., for a 1% antioxidant use level, 8.9 mg per sample of antioxidant is added). This antioxidant amount can be added to the solvent system along with the squalene and the bacterial metabolite (which is optional).

The Oxidizable Component should be mixed in a solvent system for the method described herein. Although it is possible to test the Oxidizable Component in the absence of a solvent, using a solvent is desired for ease and repeatability. In some aspects, the solvent can be aqueous or can be nonaqueous, and should be present in an amount sufficient to reliably dissolve the Oxidizable Component to a sufficient level. For the examples set forth herein, the solvent was nonaqueous. The volumetric amount of solvent(s) used can be from about 5 to about 20 times the volumetric amount of the Oxidizable Component, or can be from about 5 to about 10 times the volumetric amount of the Oxidizable Component. Suitable solvents can include one or more of the following: dimethyl sulfoxide (DMSO), glycols such as propylene glycol, 1-butanol, and combinations thereof. It is desired that the solvent system used be sufficient to dissolve at least 99% of the Oxidizable Component.

The present disclosure can include a simple solvent system that can be used to solubilize a number of different components, including the Oxidizable Component as well as antioxidants to be tested. It can be desired that various types of antioxidants be tested concurrently, including, for example, hydrophilic antioxidants and lipophilic antioxidants, among others, and the use of a consistent solvent system for all tests would be useful in giving a direct comparisons of antioxidant efficacy. Use of differing solvents used within the same test assay could result in convoluted data by use of varying solvent compositions, some of which can exhibit antioxidant activity (i.e., DMSO).

If desired, particularly when examining the oxidation of a biological Oxidizable Component, a bacterial metabolite can be used as part of the sample tested. Suitable bacterial metabolites include, for example, protoporphyrins such as protoporphyrin IX. The bacterial metabolite can be used in any desired amount, including from about 0.01 to about 2.0 nM, or about 0.1 to about 0.6 nM. The preferred amount would be a clinically relevant amount that has been observed in skin.

The antioxidants to be screened are then selected, and desired amounts of antioxidants be determined. As noted, any component that has or can have antioxidant properties can be tested in the present inventive method, in any desired amount relative to the Oxidizable Component. Exemplary antioxidants that can be tested include, for example, vitamins (e.g., vitamins C and E), natural extracts containing phenolic antioxidant compounds (e.g., flavonoids, catechins, carotenoids, anthocyanins), and synthetic antioxidants.

The antioxidants used can individually be combined with the solvent, either before or after the Oxidizable Component is added to the solvent. Further, the antioxidant and solvent can be combined in any desired way, and can be heated or vortexed to aid in combining the antioxidant and solvent.

Individual samples to be tested are then prepared, which include, for each sample, the Oxidizable Component target, the solvent, the antioxidant, and optionally a bacterial metabolite. The amount of Oxidizable Component in each sample should remain substantially consistent. For example, in some aspects, each sample includes approximately 25 μg/cm². As used herein, the term “substantially” means that the amount in each sample does not vary by more than about 5% from another sample or more than about 1% from another sample. The desired amount of antioxidant, solvent, and optionally, the bacterial metabolite, are added to the sample. Any number of samples can be prepared, where each sample differs in the amount or type of antioxidant. The solvent, the Oxidizable Component, and the optional bacterial metabolite should be substantially consistent among each of the samples. It is important that at least two samples be control samples, which includes the same amount of solvent, Oxidizable Component, and optional bacterial metabolite as the other samples, but has no added antioxidant. One control sample can be UV-stimulated, along with the samples containing antioxidants, while the other may not be UV-stimulated.

An assay is prepared, including each sample in its own well or other sampling device. Any number of wells can be used as desired. Once the desired number of samples is prepared, the samples are then subjected to the oxidation test, which can include exposure to a certain amount and time of UV radiation. The level and time of UV exposure desired to be tested is determined. The intensity and length of time of exposure can vary depending upon the intended test. Higher intensity UV exposure can be required, for example, to determine the effectiveness of the antioxidant in regions where the sun's rays are stronger, or to determine the effectiveness of the antioxidant in a time of year where the sun's rays are stronger. The length of time of UV exposure can be varied to account for the length of time that an individual would be likely to be outdoors with the antioxidant applied to the skin. In some aspects, the desired exposure can be 20 J/cm² of UVA rays, which is considered to be substantially equivalent to 2 hour exposure to intense sunlight. The exposure dose can be measured by a UVA detector to determine the cumulative dose during the exposure, and the UV generating source can be turned off when the target dose has been applied to the sample. The exposure time can vary as needed or desired to give a target dose of UV radiation, which can vary from about 10 J/cm² to about 30 J/cm², or about 20 J/cm². Ultimately, it is desired to test the samples at a target dose of radiation exposure (rather than a certain intensity for a certain length of time), where the target dose of radiation exposure is equivalent to the intended target sun exposure.

After the samples have been subjected to the desired amount of radiation for the length of time desired, the samples can be removed from the exposure and analyzed. Specifically, the amount of the oxidized byproduct in each sample is measured. Measurement can be by any desired means, and can include analysis chemically, physically, or through other means. In one aspect, the measurement can be achieved through fluorometric analysis. In one particular method, a TBARS assay is useful to determine the amount of MDA, the lipid peroxidation byproduct, generated in each sample. Comparing the amount of oxidation byproduct in each sample can therefore allow the user to determine the efficacy and efficiency of the particular sample tested. Specifically, the type and amount of antioxidant as compared to the amount of Oxidizable Component tested can be determined. The control samples, which had no added antioxidant, will produce a baseline level of oxidized byproduct upon which the tested samples can be compared using appropriate statistical methods.

When comparing the amount of oxidization byproduct in a given sample compared to the amount of byproduct generated in the radiation-exposed control sample, efficacy of the antioxidant used is determined by a statistically significant reduction in the oxidized byproduct. Statistically significant can differ in the actual amount of reduction in the formation of the oxidization byproduct, and in some aspects, the amount of oxidization byproduct in a sample can be less than the amount of oxidization byproduct in the radiation-exposed control sample by at least 5%, or at least 10%, or at least 15%, or at least 20%, or at least 25% to be considered significant.

The Oxidizable Component tested can be a biological substance, such as squalene. As noted above, one particular byproduct of squalene oxidation is MDA. It is desired that the sample containing the antioxidant(s) significantly reduce the amount of MDA formed as compared to the amount of MDA formed by the untreated, control sample. As noted, a significant reduction in the formation of the oxidation byproduct can be a reduction of at least 5%, or at least 10%, or at least 15%, or at least 20%, or at least 25% compared to the untreated control sample.

Once the antioxidant type and amount of antioxidant that is effective and efficient to protected against oxidation of a particular Oxidizable Component is determined, a composition including the antioxidant in the desired amount can be prepared. As noted, the composition can include the antioxidant and the Oxidizable Component, where the antioxidant is useful to protect against oxidation of the Oxidizable Component in the composition. Alternatively, the composition can include the antioxidant, where the composition is intended to be used in a fashion where the composition comes into contact with the Oxidizable Component, and therefore the application of the composition can be effective in reducing oxidation of the Oxidizable Component. For example, the composition can be a topical composition, such as a moisturizer or sunscreen, and the Oxidizable Component can be squalene. Application of the composition, including the effective and efficient amount and type of antioxidant, can therefore help reduce the likelihood of squalene oxidation on the skin of the user. As noted above, reduction in squalene oxidation can help reduce the likelihood of clogging pores and acne generation.

Therefore, the present disclosure can include a method of forming a composition including an effective and efficient level of an antioxidant, where the composition is effective at reducing the oxidation of an Oxidizable Component. The composition can include the Oxidizable Component therein, or it can be applied to a surface that includes an Oxidizable Component. The Oxidizable Component when from natural source can also increase with time being constantly produced by the skin. The antioxidant composition can therefore serve as a long-term protection being present on the skin, even as the Oxidizable Component is being produced while the composition is applied to the skin. For example, squalene is constantly produced on the skin, and therefore the amount of squalene produced while the composition is applied to the skin can vary. The present disclosure allows for a method of use of a composition that provides desired antioxidant effect even when additional components are produced naturally. The method can include the steps of performing the test outlined herein, identifying a type and amount of antioxidant that is determined to be effective and efficient in reducing oxidation, and forming a composition including the identified antioxidant in the amount determined to be effective and efficient.

The present disclosure also can include a method of using a composition including effective and efficient level of an antioxidant, where the composition is applied to a surface including an Oxidizable Component, and the composition is effective at reducing oxidation of that Oxidizable Component. The composition can be applied to the skin, and the Oxidizable Component can be a component found in or on the surface of the skin, such as squalene. In such an embodiment, the composition can be effective at reducing the likelihood of acne and clogged pores, due to reduction in the oxidation of squalene, as discussed above. Thus, various methods can be used as described herein, including methods of screening antioxidants, methods of preparing compositions, methods of using compositions, and methods of reducing formation of acne.

### Examples

The following Examples are intended to illustrate, but not to limit, the disclosed subject matter in any manner, shape, or form, either explicitly or implicitly.

The Oxidizable Component tested was squalene, with the oxidation byproduct being malondialdehyde (MDA). Solvents were first tested to determine if certain solvent systems would be acceptable to solubilize the Oxidizable Component. First, ethanol and acetonitrile were used, but these solvents were not able to solubilize the squalene to a desired level. Then, 30% propylene glycol (PG) was added to ethanol, so as to increase lipophilicity, which initially appeared to solubilize squalene with vigorous mixing. However, the squalene was found to later coalesce and float as large droplets on the solvent. This immiscibility would not enable accurate measurements or give relative comparisons of antioxidant efficacy.

The antioxidant test model described above was used to test efficacy of various antioxidants in reducing the oxidation of squalene in conditions reflecting approximately two (2) hours of intense sun exposure. The target Oxidizable Component was squalene, in an amount of 30 μL (9.7% w/w) per sample. The solvents selected were dimethyl sulfoxide, propylene glycol (PG), and 1-butanol, in amounts of 8.3% (v/v), 8.3% (v/v), and 83.5% (v/v) per sample, respectively. The tests further included a biological metabolite, specifically protoporphyrin IX, in an amount of 0.12 nM per sample. The exposure to UVA radiation, using a solar simulator (NewportR), Oriel Instruments U.S.A., model 91293-1000), was 20 J/cm², as measured by a calibrated UVA detector probe (Solar Light Company, Inc., model PMA2110-UVS) connected to a calibrated simulator controller (Solar Light Company, Inc., model DCS-2). Several antioxidants were tested, across a study panel, set forth in Table 2 below.

To prepare the antioxidant test solutions, the antioxidants were first dissolved (via vortexing and gentle heating if necessary) in a volume of dimethyl sulfoxide (DMSO) that achieved a target concentration for the predetermined use level as outlined in Table 1.

TABLE 1
Concentrated test solution concentrations
for antioxidants dissolved in DMSO

| Predetermined | Weight of ingredient | Concentrated test |
|---|---|---|
| antioxidant use level | (mg) | solution concentration |
| (% w/w) in the formula | per well | (mg/mL) |
| 0.01 | 0.08928 | 4 |
| 0.05 | 0.4464 | 20 |
| 0.1 | 0.8928 | 40 |
| 0.5 | 4.464 | 200 |
| 1 | 8.928 | 400 |
| 2 | 17.856 | 800 |
| 3 | 26.784 | 1200 |

The antioxidant test solutions were then diluted by 50% with propylene glycol (PG). A bulk volume (enough for 7 replicates to have excess for completion of 5 replicates) for each test article was prepared in amber glass vials by combining 312.5 μL of the target antioxidant/DMSO/PG solution (control samples included no antioxidant) with 1577.5 μL of 1-butanol containing 0.16 nM protoporphyrin IX (0.12 nM concentration in mixture), and 210 μL of squalene. The bulk volume was vortexed immediately prior to pipetting 5 replicates of 300 μL samples into the wells of a 24-well plate. The plates were then exposed to the UVA radiation until the target UVA dose was achieved (20 J/cm²), after which the plate was removed and the amount of MDA formed by lipid peroxidation was analyzed. Care was taken during the experiment to protect the samples from extraneous light oxidation throughout the solution preparation, sample plating, and after the UVA exposure prior to MDA quantitation.

The determination of the amount of MDA formed was performed by fluorometric analysis using a commercial TBARS assay kit (Sigma Aldrich, catalogue #MAK085) that reacts thiobarbituric acid with the MDA present in the sample to form a fluorometric product (λex=532/λem=553 nm), which can be easily quantitated with a monochromator-based microplate reader (Tecan Infinite M1000 Pro) and the accompanying software (Tecan i-control). Efficacy was determined by performing statistical analyses (1-Way ANOVA) comparing the measured MDA product fluorescence of the UVA-stimulated, untreated control to the treated samples and determining if a statistically significant (p<0.05) reduction was observed. This statistical comparison also included comparing an untreated control sample that was not exposed to UVA to one that was exposed to UVA to confirm a significant increase in oxidation as a result of the UVA exposure. The magnitude of efficacy for each sample, expressed as percentage reduction from the untreated control, was averaged for each antioxidant treatment.

Results are shown in Table 2 below, with the level of MDA generated being determined by fluorescence intensity which is proportional to the amount of MDA generated in the sample. The results designated as “Efficacy” show the percentage difference when compared to the untreated, UV-stimulated control sample. Control samples were prepared and evaluated at various times, with the % change from a UV stimulated control sample being −63%, −65%, −24%, −34%, −17%, and −51%. It is expected that the difference in efficacy of a control sample that has not been exposed to UV radiation compared to one that has been exposed to UV radiation would be between −50 to −65%. Control tests in which there was a lower difference (e.g., higher oxidation in the untreated, non-UV stimulated control) can be attributable to opening the test vial a few times to plate the UV stimulated control, or from placing in centrifuge tubes first (for subsequent TBA solution addition) and being open to air during the time it takes to pipet the many other samples. In Table 2 below, “X” means that there was no significant reduction (p<0.05) in the MDA formation as compared to the UV-stimulated control. The % of concentration of the active in the formula tested is identified as “% conc”.

In addition to the samples identified in Table 2 below, sodium ascorbyl phosphate (in a 1% concentration) was also evaluated. It demonstrated an efficacy of −41 percentage change from the UV stimulated control.

TABLE 2
Study Panel MDA generation

| Sample | | Sample | | Sample | |
|---|---|---|---|---|---|
| (% conc) | Efficacy | (% conc) | Efficacy | (% conc) | Efficacy |
| Vitamin E | −61 | Resveratrol | −42 | 1:1 Glycolic, | X |
| (alpha- | | (0.1%) | | Lactic acid (6%) | |
| tocopherol) | | | | | |
| (0.1%) | | | | | |
| Vitamin E | −78 | p-coumeric acid | X | Mandelic acid | 81 |
| (alpha- | | (0.01%) | | (2%) | |
| tocopherol) | | | | | |
| (0.5%) | | | | | |
| Vitamin E | −81 | p-coumeric acid | −48 | Mandelic acid | 69 |
| (alpha- | | (0.05%) | | (3%) | |
| tocopherol) (1%) | | | | | |
| Vitamin E acetate | −36 | p-coumeric acid | −55 | trans-cinnamic | X |
| (0.1%) | | (0.1%) | | acid (0.1%) | |
| Niacinamide | X | Ferulic acid | −47 | gallic acid (0.1%) | X |
| (0.01%) | | (0.01%) | | | |
| Niacinamide | X | Ferulic acid | −41 | Feverfew (0.1%) | X |
| (0.1%) | | (0.05%) | | | |
| Niacinamide | 58 | Ferulic acid | −39 | Feverfew (1%) | X |
| (0.5%) | | (0.1%) | | | |
| D-Panthenol | X | Symdecanox HA | X | Beta-carotene | X |
| (Provitamin B5) | | (0.5%) | | (1%) | |
| (0.1%) | | | | | |
| D-Panthenol | −21 | Symdecanox HA | −41 | Rosemary (1%) | X |
| (Provitamin B5) | | (1%) | | | |
| (0.25%) | | | | | |
| D-Panthenol | −18 | Glycolic acid | −25 | Caraway Seed | X |
| (Provitamin B5) | | (3%) | | (1%) | |
| (0.5%) | | | | | |
| Green Tea | −20 | Glycolic acid | X | Aloe Vera | X |
| extract (0.1%) | | (6%) | | (0.01%) | |
| Green Tea | −25 | Butylated | −54 | Aloe Vera (0.1%) | X |
| extract (1%) | | hydroxytoluene | | | |
| | | (BHT) (0.035%) | | | |
| Dragocalm (oat | X | Lemon Aspen | −43 | Aloe Vera (0.5%) | X |
| extract) (1%) | | extract (1%) | | | |
| Dragocalm (oat | −27 | Lemon Aspen | −30 | Dragon's Blood | X |
| extract) (3.5%) | | extract (1%) | | (0.1%) | |
| OiLESS'city ™ | −51 | Lemon Aspen | −25 | Dragon's Blood | X |
| Lemon Myrtle | | extract (1%) | | (0.3%) | |
| Extract (1%) | | | | | |
| OiLESS'city ™ | −49 | Farnesyloxy | −27 | Polygonum | −35 |
| Lemon Myrtle | | phenylpropanoid | | cuspidatum | |
| Extract (2%) | | (0.01%) | | (0.1%) | |
| Apigenin (0.5%) | −35 | Farnesyloxy | −30 | Polygonum | −77 |
| | | phenylpropanoid | | cuspidatum | |
| | | (0.05%) | | (0.5%) | |
| Polygonum | −35 | Farnesyloxy | −22 | Polypodium | −15 |
| cuspidatum | | phenylpropanoid | | leucotomos | |
| (0.1%) | | (0.1%) | | (0.1%) | |
| Polygonum | −77 | Gluconolactone | X | Polypodium | −18 |
| cuspidatum | | (1%) | | leucotomos | |
| (0.5%) | | | | (0.5%) | |
| Polypodium | −15 | Lactic acid (3%) | X | Lemon aspen | X |
| leucotomos | | | | extract batch1 | |
| (0.1%) | | | | (0.1%) | |
| Polypodium | −18 | Lactic acid (6%) | X | Lemon aspen | 10 |
| leucotomos | | | | extract batch1 | |
| (0.5%) | | | | (0.5%) | |

TABLE 2
(Cont.) - Study Panel 1 MDA generation

| Sample | | Sample | |
|---|---|---|---|
| (% conc) | Efficacy | (% conc) | Efficacy |
| Lemon aspen | 13 | Eosidin (1%) | −19 |
| extract batch1 | | | |
| (0.5%) | | | |
| Lemon aspen | X | Eosidin (0.5%) | −25 |
| extract batch1 | | | |
| (1%) | | | |
| Lemon aspen | X | BioDTox (1%) | −25 |
| extract batch2 | | | |
| (1%) | | | |
| Edelweiss (3%) | −39 | BioDTox (0.5%) | −22 |
| Edelweiss (1%) | −46 | BioDTox (1%) | −12 |
| Edelweiss | −25 | Redsnow (0.5%) | −35 |
| liposomes (1%) | | | |
| UPCYCLED | −65 | Redsnow (1%) | −46 |
| tangerine peel | | | |
| extract (0.01%) | | | |
| UPCYCLED | −59 | Redsnow (0.5%) | −48 |
| tangerine peel | | | |
| extract (0.002%) | | | |
| Chamomile | −45 | Quercevita (1%) | −82 |
| extract (0.01%) | | | |
| Chamomile | X | Quercevita (0.5%) | −84 |
| extract (0.002%) | | | |
| Grapefruit extract | −27 | Sea Kale (2%) | −46 |
| (0.01%) | | | |
| Grapefruit extract | −33 | Sea Kale (1%) | −42 |
| (0.002%) | | | |
| Perilla seed oil | 14 | Broccoli seed | −63 |
| (2%) | | extract (1%) | |
| Parsley leaf | −56 | Broccoli seed | −38 |
| extract (2%) | | extract (0.1%) | |
| Irish moss (1%) | −39 | Broccoli seed oil | −7 |
| | | (2%) | |
| Black cumin seed | −20 | Broccoli seed oil | −20 |
| oil (2%) | | (1%) | |
| Mangosteen (2%) | −21 | Thyme extract (2%) | −33 |
| Sea buckthorn | X | Thyme extract (1%) | −39 |
| juice (3%) | | | |
| Sea buckthorn | X | | |
| juice (1%) | | | |
| Skinceuticals | −73 | | |
| Silymarin CF | | | |
| Eosidin (2%) | −22 | | |

Certain samples tested were found to reduce squalene oxidation through the test methods, and certain samples were found to have an opposite impact on oxidation. Further, certain samples showed greater reduction in oxidation when a higher amount was used, while other samples showed less reduction in oxidation when a higher amount was used. This test can be useful in showing not just the efficacy of a potential antioxidant, but the amount that can be used to provide a more desired reduction in oxidation. While one sample can be likely to provide a reduction in the oxidation of squalene, it can vary in the optimal concentration to have the desired effect. Through the innovative methods described herein, a product can be developed including not only the antioxidant but also the amount of antioxidant to provide a beneficial impact on oxidation reduction. The method can include the steps above for identifying the oxidation reduction for a given antioxidant in a given amount, and can further include the step of preparing a product including that antioxidant in that amount.

The results of the in vitro test for 1% sodium ascorbyl phosphate were compared to published results of a study of human subjects (n=20), who were given a topical composition (oil-in-water emulsion) including 1% sodium ascorbyl phosphate, which is an antioxidant having anti-acne efficacy. The study was published in the International Journal of Cosmetic Science: “Sodium ascorbyl phosphate shows in vitro and in vivo efficacy in the prevention and treatment of acne vulgaris”, by Klock, et al. (May 6, 2005). In the published study, the formulation was applied twice daily to the back of the human subjects at a dose of 2 mg/cm² for 7 days prior to irradiation with 10 J/cm² UVA, collection of sebum by solvent extraction, and the amounts of squalene and squalene hydroperoxide being determined by HPLC.

As shown herein, the in vitro model demonstrated that 1% sodium ascorbyl phosphate reduced UV-induced squalene oxidation by 41% as compared to the control sample when exposed to 20 J/cm² UVA. The published study indicated that 1% sodium ascorbyl phosphate reduced UVA-induced squalene oxidation by 30%, which was significant (p<0.05 as determined by a t-test) when compared to a placebo formulation. The results obtained through the inventive method described herein are considered to be comparable to the in vivo results of the published study referenced above, considering the lower dose of UVA used in the published study (10 J/cm²) compared to the in vitro study (20 J/cm²).

The results shown herein provide that the methodology is effective to identify the amount of protection that various components can give against squalene oxidation, which can then be used to prepare one or more products including that antioxidant in a given amount.

1. A method of screening one or more antioxidants in the reduction of oxidation of an oxidizable component, comprising:
preparing one or more test samples comprising the oxidizable component, the one or more antioxidants, a solvent, and optionally one or more bacterial metabolites,
preparing one or more control samples without the one or more antioxidants and comprising the oxidizable component, the solvent, and optionally the one or more bacterial metabolites,
performing an oxidation test on each of the samples,
measuring an amount of an oxidation byproduct of each of the samples, and
determining a level of oxidation mitigation by comparing the measured amount of the oxidation byproduct in the one or more test samples to the measured amount of the oxidation byproduct in the one or more control samples.

2. The method of claim 1, wherein the oxidizable component includes squalene.

3. (canceled)

4. The method of claim 1, wherein the samples include the oxidizable component in an amount of about 30% (v/v) or less.

5. The method of claim 1, wherein the solvent comprises dimethyl sulfoxide, propylene glycol, 1-butanol, or combinations thereof.

6. The method of claim 5, wherein the solvent comprises dimethyl sulfoxide in an amount of from about 5% (v/v) to 15% (v/v), propylene glycol in an amount of from about 5% (v/v) to about 15% (v/v), and 1-butanol in an amount of from about 70% (v/v) to about 90% (v/v).

7. (canceled)

8. The method of claim 1, wherein the oxidation byproduct includes malondialdehyde (MDA).

9. The method of claim 1, wherein the measured amount of the oxidized byproduct in the one or more test samples is at least about 10% less than the measured amount of the oxidized byproduct in the one or more control samples.

10. (canceled)

11. The method of claim 1, wherein the oxidation test includes exposing the samples to an oxidation-inducing element.

12. (canceled)

13. (canceled)

14. The method of any of claim 1, wherein the one or more bacterial metabolites comprises protoporphyrin IX, coproporphyrin I, coproporphyrin III, or combinations thereof.

15. (canceled)

16. The method of claim 1, wherein the amount of the oxidation byproduct is measured by fluorometric analysis.

17. A method of determining efficacy of one or more antioxidants in reducing or mitigating oxidation of an oxidizable component, comprising:
selecting a type and an amount of the oxidizable component as a target,
selecting one or more antioxidants for testing against the target,
selecting a solvent,
optionally selecting one or more bacterial metabolites,
combining the oxidizable component, the one or more antioxidants, the solvent, and optionally the one or more bacterial metabolites, to form a test sample,
performing an oxidation test on the test sample,
measuring an amount of an oxidation byproduct of the test sample, and
determining a level of oxidation mitigation by comparing the measured amount of the oxidation byproduct of the test sample to a control sample without the one or more antioxidants.

18. The method of claim 17, wherein the oxidizable component includes squalene.

19. The method of claim 17, wherein the samples include the oxidizable component in an amount of from about 1% (v/v) to about 30% (v/v).

20. The method of claim 17, wherein the solvent comprises dimethyl sulfoxide, propylene glycol, 1-butanol, or combinations thereof.

21. (canceled)

22. The method of claim 17, wherein the oxidation byproduct includes malondialdehyde (MDA).

23. The method of claim 17, wherein the oxidation test includes exposing the samples to a predetermined amount and a predetermined time of UV radiation.

24. (canceled)

25. The method of claim 17, wherein the measured amount of the oxidization byproduct in the test sample is less than the measured amount of the oxidization byproduct in the control sample by at least 5%.

26. The method of claim 17, further comprising preparing a composition including the one or more antioxidants determined to be effective in reducing or mitigating oxidation of the oxidizable component.

27. A method of using a composition including one or more antioxidants, comprising applying the composition to a surface including an oxidizable component, wherein the composition is effective at reducing oxidation of the oxidizable component.

28. (canceled)

29. (canceled)

30. The method of claim 27, wherein the composition reduces the formation of clogged pores and acne.
